(19) United States
(12) Patent Application Publication
(10) Pub. No.: US 2025/0151730 A1
(43) Pub. Date: May 15, 2025

(54) MIXTURE OF ISOLATED BACTERIA OF CURCUMA LONGA

(71) Applicants: HER MAJESTY G. K. CHOEDEN WANGCHUCK'S TRUST FUND, Thimphu (BT); ACTINA FZCO, Dubai (AE)

(72) Inventors: Her Majesty Ashi Kesang Choeden WANGCHUCK, Thimphu (BT); Mizuho NASU, Tokyo (JP); Bejit IDEAS, Tokyo (JP); Dan Lifton, Purchase, NY (US)

(21) Appl. No.: 18/838,895

(22) PCT Filed: Feb. 15, 2023
(86) PCT No.: PCT/IB2023/051371

§ 371 Date: Aug. 15, 2024

(30) Foreign Application Priority Data
Feb. 15, 2022 (FR) FR2201333

(51) Int. Cl. A01N 63/20; A01P 21/00; C12N 1/20

(52) U.S. Cl. CPC A01N 63/20; A01P 21/00; C12N 1/20

(57) ABSTRACT

The present invention relates to a plant treatment composition comprising a mixture of live bacteria, bacterial lysate and metabolites from the rhizomes of Curcuma longa. Spraying or applying this composition to plants increases plant fertility and anti-inflammatory properties of cereals, fruits and vegetables.

### FIELD OF THE INVENTION

The subject of the present invention is a plant treatment composition intended to increase the yield of agricultural production such as cereals, fruits and vegetables. Advantageously, the treatment according to the invention makes it possible to increase the beneficial properties of agricultural production on human health. The treatment composition according to the invention may contain a mixture of live bacteria originating from the extraction of the microbiota of the rhizomes of turmeric Curcuma longa, preferably originating from Bhutan, a lysate of these bacteria and/or metabolites originating from the slow fermentation of these same bacteria. Advantageously, the bacteria according to the invention persist sustainably in the soil.

### BACKGROUND OF THE INVENTION

Just like humans, plants have a microbiome that is composed of bacteria, yeasts, fungi or viruses that colonize all accessible plant tissues. These microorganisms can form complex co-associations with plants and play an important role in promoting plant health in the natural environment. The plant microbiota is made up of numerous and varied species of microorganisms, which provide several essential functions to the plant. The microbiota forms, for example, a symbiotic relationship with the roots of plants that allow them to both benefit from their environment and enrich it. The root system of plants explores the soil in order to find nutrients and is therefore exposed to the various microorganisms present in the soil. Plants provide sugar to microorganisms, while microorganisms provide nitrogen and phosphorus, essential for the proper development of plants. Microorganisms also produce plant hormones that stimulate plant growth.

The microbial population can be altered by the environment and agricultural methods, particularly by intensive agriculture, monoculture and the massive use of agrochemicals such as herbicides, fungicides, insecticides, nematicides, molluscicides, rodenticides, chemical fertilizers. The use of chemicals significantly decreases the diversity and composition of the soil and plant microbiome. This alteration of the diversity and composition of the beneficial microbial community can be unfavorable to plant growth and development either by reducing the availability of nutrients or by increasing the incidence of diseases (Meena, R. S. et al. “Impact of Agrochemicals on Soil Microbiota and Management. A Review”. Land 2020, 9, 34). The alteration of the microbial community can also impact plant fertility and the yield of seeds, fruits and plant matter. Microbes play a key role in ecosystems and influence a large number of important ecosystem processes, including plant nutrient acquisition, nitrogen and carbon cycling, and soil formation and composition. In particular, the soil microbiome forms a symbiosis with plants that is essential for nutrient uptake and growth. Fertilizer solutions containing bacteria of the genus Bacillus (WO2017105238A1) or Bacillus, Pseudomonas, (WO2015118516A1) have been proposed to fertilize the soil and allow faster plant growth. However, their actions are limited. Indeed, these products are unique bacteria from a different environment that cannot compete with the indigenous microbiome that has already formed a stable and balanced ecosystem. The study by E. S. Jensen and L. H. Sørensen (“Survival of Rhizobium leguminosarum in soil after addition as inoculant”, FEMS Microbiology Ecology, Volume 3 (4), August 1987, Pages 221-226), shows that the population of bacteria inoculated into the soil decreases with time and that the half-life of an inoculant is about 1.5 to 2.1 years. This shows that plant treatment solutions containing only bacteria are not sustainable solutions and require continuous application, usually every year, to have a significant effect, resulting in a waste of time and money. Similarly, while existing solutions improve plant growth, they do not impact the benefits of these plants on human health.

It was by studying turmeric bacteria that the Applicant discovered, quite unexpectedly, that these problems can be easily solved by using a composition intended to increase the yield of agricultural production containing a mixture of live bacteria, a lysate of bacteria and/or metabolites originating from the extraction of the microbiota of turmeric rhizomes (Curcuma longa), preferably Curcuma longa cultivated in Bhutan.

### SUMMARY OF THE INVENTION

The invention relates to a plant treatment composition comprising a mixture of live bacteria from the microbiota of the rhizomes of turmeric (Curcuma longa), preferably from Bhutan, a lysate of said bacteria and metabolites from the fermentation of these same bacteria. This composition is intended to increase the yield of agricultural production, preferably cereals, fruits and vegetables, and to increase their beneficial properties on human health. The plant treatment composition according to the invention may comprise one or more strains of bacteria selected from the following: Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus para casei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, Bradyrhizobium elkanii, Bradyrhizobium japonicum, Gluconacetobacter diazotrophicus, Azospirillum brasilense.

The plant treatment composition may be in the form of liquids, foams, pastes, emulsions, oils, gel, jellies, syrups, solids, powders, sprays, or aerosols.

The invention also relates to a method for manufacturing the plant treatment composition as described below, comprising the steps of:
isolating bacteria from Curcuma longa rhizomes, preferably from a plantation located in Bhutan and not using chemical fertilizers or herbicides; then
inoculating at least one substrate with said bacteria and fermenting the mixture of bacteria with said at least one substrate.

According to another aspect, the invention relates to the use of the plant treatment composition according to the invention for:
increasing soil fertility by increasing the concentration of nutrients and decreasing the amount of toxic substances.
increasing the yield of cereals, fruits and vegetables, by increasing plant growth, their fertility and by increasing the number and weight of seeds, fruits and vegetables.
to increase the beneficial properties of cereals, fruits and vegetables on human health.
to increase the anti-inflammatory properties and the blood sugar regulating properties of cereals, fruits and vegetables.
to increase the dietary fibre content of cereals, fruits and vegetables.

According to a final aspect, the invention relates to a method for treating plants intended to increase the yield of at least one plant, in which the composition according to the invention is sprayed or spread on the soil and/or the plant.

### DETAILED DESCRIPTION OF THE INVENTION

The subject matter of the present invention therefore relates to a plant treatment composition intended to increase the yield of agricultural production. The invention also relates to the use of the composition, as described below, for treating at least one plant, preferably a plant intended for agricultural production. In one embodiment, the agricultural production designates cereals, fruits and/or vegetables. In a specific embodiment, the agricultural production designates cereals, preferably rice. In a specific embodiment, the agricultural production designates fruits, preferably apples.

According to one embodiment, the treatment composition makes it possible to increase the beneficial properties of the agricultural production on human health.

The composition may comprise a mixture of live bacteria from the microbiota of the rhizomes of fermented turmeric (Curcuma longa), preferably originating from Bhutan, a lysate of bacteria and metabolites originating from the extraction.

In a first embodiment, the composition comprises a mixture of live bacteria from the microbiota of the rhizomes of turmeric (Curcuma longa), preferably from Bhutan. Advantageously, the bacteria applied according to the invention have a long-lasting persistence in the soil.

In a second embodiment, the composition comprises a lysate of the bacteria from the microbiota of the rhizomes of turmeric (Curcuma longa), preferably turmeric from Bhutan.

In a third embodiment, the composition comprises metabolites from the extraction of fermented bacteria from the microbiota of the rhizomes of turmeric (Curcuma longa), preferably turmeric from Bhutan.

In a preferred embodiment, the composition comprises or consists of the constituents of the three aforementioned embodiments.

The composition can be used in increasing soil fertility. The invention also relates to a composition characterized in that it is used in the modulation of human blood sugar by increasing the amount of dietary fiber in cereals, fruits and vegetables thus cultivated.

According to one embodiment, the use of the composition of the invention increases the growth and/or yield of plants. It should be noted that the composition is characterized in that the vaporization or application of said composition on the plants or on the soil surrounding the plants increases the growth and yield of the plants. The invention also relates to a composition characterized in that the vaporization or application of said composition on the plants advantageously allows its use in increasing the beneficial properties on the health of the consumer of the cereals, fruits and vegetables thus cultivated. In one embodiment, the use of the composition of the invention increases the yield of plants in nutrients beneficial to human health such as fiber, vitamins, minerals, polyphenols, terpenes, nitrogen compounds, alkaloids, steroids, terpenoids, flavonoids, omega-3, omega-6 and omega-9, linoleic acid, L-carnitine, choline or sphingomyelin. In one embodiment, the use of the composition of the invention increases the yield of plants in nutrients beneficial to human health such as vitamins, minerals, polyphenols, omega-3, omega-6 and omega-9 and/or linoleic acid. In a specific embodiment, the use of the composition of the invention increases the yield of plants in fiber. For the clarity of the following explanations, we decide to call QKC a composition intended to increase the yield of plants and increase their beneficial properties on human health containing a mixture of live bacteria, bacterial lysate and metabolites from the extraction of the microbiota of the rhizomes of turmeric (Curcuma longa), preferably from Bhutan.

The applicant describes below a composition intended to increase the yield of plants and increase their beneficial properties on human health containing a mixture of live bacteria, bacterial lysate and metabolites from the extraction of the microbiota of the rhizomes of turmeric (Curcuma longa), preferably from Bhutan.

Microorganisms have long been applied as inoculants for biological control or biostimulation. However, these microorganisms have a limited effect. Indeed, the products containing these microorganisms most often contain bacteria from a unique and different environment. Which induces an irregular performance varying according to the climate, the type of soil and other factors. Indeed these applied bacteria cannot compete with the indigenous microbiome. The functionality and persistence of microorganisms depend on the interactions with the environment as well as with other microorganisms within a community. The applicant has therefore developed a plant treatment composition containing both bacteria, a lysate of bacteria as well as fermentation metabolites thereof. The metabolites make it possible both to modulate the microbiota already existing in the soil as well as to allow the bacteria to persist disrupted in the soil by modulating the communication between the bacteria of the invention and the bacteria of the soil by modulating quorum sensing. Quorum sensing is the communication mechanism of bacteria allowing one strain of bacteria to inhibit or stimulate growth as well as to modulate the gene expression of another strain of bacteria. The present invention therefore describes a plant treatment composition intended to increase the yield of agricultural production of cereals, fruits and vegetables and to increase their beneficial properties on human health, characterized in that it contains a mixture of live bacteria, bacterial lysate and metabolites originating from the extraction of the microbiota of the rhizomes of turmeric (Curcuma longa), preferably originating from Bhutan and having an increased persistence in the soil.

The applicant describes below a means of obtaining the plant treatment composition containing a mixture of live bacteria, bacterial lysate and metabolites originating from the extraction of the microbiota of the rhizomes of turmeric, preferably turmeric (Curcuma longa) originating from Bhutan.

According to another aspect, the invention relates to a method for preparing the composition according to the invention.

In one embodiment, the method is in several steps comprising:
a step of extracting bacteria from Curcuma longa rhizomes, preferably from a plantation located in Bhutan and not using chemical fertilizers or herbicides,
a step of selection by PCR followed by sequencing of the 16s rRNA, and
a step of fermentation in the presence of substrate.

According to one embodiment, the method comprises the steps of
isolating bacteria from Curcuma longa rhizomes, preferably from a plantation located in Bhutan and not using chemical fertilizers or herbicides;
inoculating at least one substrate with said bacteria and fermenting the mixture of bacteria with the substrate.

In one embodiment, the method is a method of preparing the composition comprising a mixture of live bacteria from the microbiota of turmeric rhizomes (Curcuma longa), preferably from Bhutan, a lysate of said bacteria, and metabolites from the extraction of said fermented bacteria; said method comprising the steps of i) collecting turmeric rhizomes, preferably grown in a field in Bhutan, even more preferably without having undergone chemical treatment; ii) isolating bacteria from the microbiome of the rhizomes; iii) culturing the isolated bacteria so as to obtain a mother culture of the bacteria; iv) inoculating a sterilized substrate with bacteria from the mother culture and fermenting, preferably in a fermentation tank for at least 120 hours at a temperature between 30° C. and 45° C.; v) sterilizing and microfiltrating the mixture fermented in step iv) giving rise to a composition comprising the fermentation metabolites and the bacterial lysates; vi) mixing the composition comprising the fermentation metabolites and the bacterial lysates with bacteria from the mother culture of bacteria isolated from turmeric. The invention also relates to a composition that can be obtained, or directly obtained, by the method according to the invention

In one embodiment, the composition according to the invention comprises from 0.5 to 10% of the mother culture.

The applicant studied turmeric rhizomes from different countries and compared their effectiveness on anti-inflammatory gene expression. It turned out that turmeric from Bhutan was more effective in inducing an anti-inflammatory effect. By studying the microbiota of this turmeric, the applicant discovered that the microbiota of Bhutanese turmeric produced small molecules, called metabolites, which gave Bhutanese turmeric its exceptional effects on health. The applicant therefore developed a method for extracting this microbiota as well as a fermentation method for producing the metabolites and bacterial lysates. First, turmeric rhizomes grown in a field in Bhutan that had not used chemicals were collected. The samples were collected and then mixed at 20% with a phosphate saline buffer at pH 7 and a concentration of 1 mol/L. After centrifugation, the supernatant was collected. The supernatant was cultured in the presence of an agent that stimulates bacterial growth. The species were identified by PCR and then 16s rRNA sequencing was performed. PCR or polymerase chain reaction is a technique for amplifying DNA fragments in vitro. DNA amplification allows easier sequencing and better species identification. 16s rRNA is an RNA fragment that all species of bacteria have with sequence variations characteristic of each species. The person skilled in the art is capable of performing 16s rRNA sequencing according to his general knowledge, as demonstrated by way of example by the materials and methods point 2.2.1 of Kumar et al. 2016 which is incorporated by reference (Ajay Kumar et al., Biocatalysis and Agricultural Biotechnology, Volume 8, 2016,

Pages 1-7). The sequences obtained for the turmeric microbiotas are compared to a ribosomal sequence database. The selected bacteria are cultured in the presence of a substrate promoting their multiplication and constitutes the mother culture of bacteria of interest.

The selected bacteria are then used in a fermentation process. This fermentation process takes place in a fermentation tank in several stages for a total duration of at least 120 hours including prior sterilization of the ingredients intended to undergo fermentation, inoculation of the bacteria and culture of the mixture at a temperature between 30° C. and 45° C.

The ingredients used as substrate during the fermentation process are all of bio-sourced and edible origin, i.e. all plants such as fruits, vegetables and algae, as well as food waste from households, workplaces and catering establishments, as well as compost and manure. The fermented substrate ingredients can be for example, but not limited to, fruits, peels, leaves or flowers of Figs, Olives, Rosemary, Thyme, Beans, Sage, Melons, Pomegranates, Soybeans, Watermelons, Cabbage, Green or Black Tea, Turmeric, Watermelon, Pine Needles. The materials are selected to be grown without the use of pesticides. A mixture of several materials without limit of number or only one can be used as a substrate for fermentation. At the end of fermentation, the contents of the fermentation tank are sterilized and filtered using microfiltration to remove plant debris and keep only the metabolites and bacterial lysates. The metabolites+lysate mixture is then mixed with the mother culture of bacteria isolated from turmeric. The composition is adjusted to contain from 0.5 to 10% of the mother culture.

The bacteria isolated from turmeric, such as for example those from the mother culture, and identified by 16s rRNA sequencing can be, without limitation: Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, obium elkanii, Bradyrhizobium japonicum, Gluconacetobacter diazotrophicus, Azospirillum brasilense. In one embodiment, the bacteria isolated from turmeric, such as for example those from the mother culture, and identified by 16s IRNA sequencing include Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, Bradyrhizobium elkanii, Bradyrhizobium japonicum, Gluconacetobacter diazotrophicus, Azospirillum brasilense. In one embodiment, the bacteria isolated from turmeric, as for example identified by 16s rRNA sequencing, include Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Archaeospora trappei, Methylobacterium crusticola, Bradyrhizobium elkanii, Bradyrhizobium japonicum, Gluconacetobacter diazotrophicus, Azospirillum brasilense. In one embodiment, bacteria isolated from turmeric, as for example identified by 16s rRNA sequencing include Christensenella minuta, Lactiplantibacillus plantarum, Lactiplantibacillus paraplantarum, Mycobacterium vaccae, Archaeospora trappei, Methylobacterium crusticola, Bradyrhizobium elkanii, Bradyrhizobium japonicum, Gluconacetobacter diazotrophicus, Azospirillum Brazilian. In one embodiment, the bacteria isolated from turmeric, such as for example identified by 16s rRNA sequencing, are chosen from Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Archaeospora trappei, Methylobacterium crusticola, Bradyrhizobium elkanii, Bradyrhizobium japonicum, Gluconacetobacter diazotrophicus, Azospirillum brasilense

In one embodiment, the method is a method for preparing the composition comprising a mixture of live bacteria from the microbiota of the rhizomes of turmeric (Curcuma longa), preferably from Bhutan, a lysate of said bacteria, and metabolites from the extraction of said fermented bacteria; said method comprising the steps of:
i) isolating the bacteria from the microbiome of the rhizomes of Turmeric, preferably grown in a field in Bhutan, even more preferably without having undergone a treatment with chemicals
ii) culturing the isolated bacteria so as to obtain a mother culture of the bacteria
iii) inoculating a sterilized substrate with bacteria from the mother culture and fermenting, preferably in a fermentation tank for at least 120 hours at a temperature between 30° C. and 45° C.; preferably said substrate being selected from materials comprising plant fibres such as fruits, vegetables and algae, as well as food waste from households, workplaces and catering establishments; preferably said substrate being selected from materials comprising plant fibres selected from fruits, peels, leaves or flowers of Figs, Olive, Rosemary, Thyme, Bean, Sage, Melon, Pomegranate, Soybean, Watermelon, Cabbage, Green or black tea, turmeric, watermelon, and pine needles;
iv) sterilising and microfiltrating the fermented mixture obtained in step iii) giving rise to a composition comprising the fermentation metabolites and the bacterial lysates;
v) mixing the composition comprising the fermentation metabolites and the bacterial lysates with bacteria from the mother culture of bacteria isolated from turmeric to give the composition comprising a mixture of live bacteria from the microbiota of the rhizomes of turmeric.

The invention also relates to a method for cultivating at least one plant, said method comprising the preparation of the composition according to the invention, in particular according to steps i)-v) above, and a step vi) comprising bringing the composition comprising a mixture of live bacteria from the microbiota of the rhizomes of turmeric obtained in step v) into contact with the cultivation soil of said at least one plant and/or with said at least one plant, preferably the composition comprising a mixture of live bacteria from the microbiota of the rhizomes of turmeric obtained in step v) being sprayed or spread on the cultivation soil of said at least one plant and/or on said at least one plant. It should be noted that the invention also relates to the use of the composition comprising a mixture of live bacteria from the microbiota of the rhizomes of turmeric, typically obtained according to steps i)-v) above for cultivating at least one plant. The method or use may be chosen from a method or use:
to increase soil fertility by increasing the concentration of nutrients and decreasing the amount of toxic substances,
to increase the yield of cereals, fruits and vegetables, by increasing plant growth, their fertility and by increasing the number and weight of seeds, fruits and vegetables;
to increase the beneficial properties of cereals, fruits and vegetables on human health; and/or
to increase the dietary fiber content of cereals, fruits and vegetables

Soil fertility is defined as the ability of a soil to support plant growth by providing essential nutrients to plants.

In particular, nitrification is an important process in soil fertility management. Nitrogen is an important element for plants. Indeed, it stimulates plant growth and regulates nutrient use. Deficiency of this element is manifested by poor leaf formation and yellowing, as well as a decrease in crop yield. However, heavy rainfall can cause nitrate to leak into the water, reducing the amount of nitrate in the soil. This nitrate leakage leads to a need for constant input of nitrate-containing fertilizer by farmers, which leads to soil acidification.

The bacteria in the QKC composition are nitrogen-fixing bacteria, that is, they are capable of fixing atmospheric nitrogen gas into “fixed nitrogen” compounds, such as ammonia, which can be used by plants.

The metabolites from the fermentation process also increase soil fertility. Indeed, these metabolites contain nutrients essential to plants. The metabolites produced can be of the following types: minerals, non-ribosomal peptides, lipopeptides, polyketides, terpenes, nitrogen compounds such as indole, pyrazines, volatile compounds containing sulfur. Terpenes can be of the following types: brassinosteroids, gibberellins. Bacterial lysates also contain elements that can improve soil fertility and provide elements necessary for plant growth.

In addition, the bacteria that make up QKC make minerals more available for absorption by plants. Plants need minerals to grow such as phosphorus, potassium, magnesium, sulfur and calcium. Minerals already exist in nature. However, their bioavailability depends on their solubility in the medium. If the minerals are in precipitated form in the soil, they cannot be absorbed by plants. The bacteria of the present invention are capable of solubilizing minerals by secreting organic acids that prevent the fixation of minerals in insoluble form. The metabolites in the composition of QKC increase the solubilization capacity of the bacteria by stimulating the genes related to this activity. The metabolites increase in particular the activity of the enzyme carbonic anhydrase, which catalyzes the dissolution of calcium carbonate.

Soils contain compounds that are toxic to both plants and human health. For example, they contain polychlorinated biphenyls (PCBs), pesticides, industrial solvents, petroleum products, dioxins and furans, heavy metals, explosives, and brominated flame retardants. These compounds can be absorbed by plants and pose a hazard to human health. Traditional technologies commonly used for the remediation of contaminated environmental soils include excavation, transportation to specialized landfills, incineration, stabilization, and vitrification. These technologies are costly and can damage soils and the landscape. Some soil bacteria have the ability to degrade toxic compounds. For example, PCBs can be degraded by dehalogenation, i.e., by removing a halogen in the PCB compound, which reduces their toxicity. However, not all bacteria have the genetic apparatus necessary to perform this degradation. The bacteria of the present invention are capable of degrading toxic compounds. The combination of bacteria and metabolites triggers the biodegradation of the toxic compounds present by stimulating the genes of the enzymes involved in the degradation of toxic chemical compounds.

The present invention is therefore described as a composition for its use in increasing soil fertility by increasing the concentration of nutrients and decreasing the amount of toxic substances.

The yield of agricultural production is defined as the amount of product harvested on a given cultivated area. It can be influenced by soil fertility, access to water as well as diseases and pests.

QKC composition increases soil fertility. QKC composition also prevents diseases by inhibiting pathogenic bacteria. As in humans and animals, plants can be infected by pathogenic bacteria and fungi. These bacteria can be of the type: Xanthomonas campestris, Clavibacter michiganensis, Pseudomonas spp., Xanthomonas campestris, Ralstonia solanacearum, Pseudomonas syringae. Phytopathogenic fungi can cause diseases such as downy mildew caused by the fungus Phytophthora infestans, fruit rot by Aspergillus, fruit rot caused by Alternaria and Botrytis. These infections can lead to fruit rot, thus leading to a decrease in harvest; they can also lead to the death of plants. Thanks to the lysozymes contained in the bacterial lysate, the QKC composition inhibits the establishment of pathogenic bacteria. Lysozymes are enzymes that destroy the membranes of pathogenic bacteria.

The metabolites of the QKC composition also stimulate the indigenous microbiota of the soil and plants, producing an additional barrier against the establishment of pathogenic bacteria.

Different strains of bacteria are able to communicate with each other through chemical signals and modulate the growth and activity of other strains by quorum sensing. Bacterial strains are also able to modulate the growth of fungi. Bacteria inhibit the proliferation of bacteria and fungi harmful to plants through competition. The metabolites of the QKC composition stimulate the indigenous microbiome and inhibit pathogenic bacteria by modulating quorum sensing.

The composition also directly increases the fertility and production of plants, especially the rice plant, by inducing a Darwinian effect, which is the activation of the autophagy gene, OsATG8a. The OsATG8a gene allows to select only the functional cells that will produce a fertile stem. Overexpression of OsATG8a significantly increases the level of autophagy and the number of fertile thalli in rice. The bacteria of the QKC composition also increase the OsNPF7.2 and OsSta2 genes involved in the number of thalli (stem bearing rice seeds) and the rice grain yield.

The present invention is therefore described as a plant treatment composition for its use in increasing the yield of cereals, fruits and vegetables, by increasing plant growth, their fertility and by increasing the number and weight of seeds, fruits and vegetables. Plants are consumed not only for their energy supply but also for their effects on health. Indeed, plants are composed of vitamins, minerals but also other molecules, such as polyphenols, terpenes, and nitrogen compounds, alkaloids, steroids, terpenoids, flavonoids, omega-3, 6 and 9, linoleic acid, L-carnitine, choline or sphingomyelin. These molecules are involved in plant growth and for resistance to biotic and abiotic stress to combat diseases and pests. These molecules also have effects on human health. Indeed, certain molecules have antioxidant properties and can also directly modulate the gene expression of human cells. In particular molecules such as curcumin, colchicine, resveratrol, capsaicin, epigallocatechin-3-gallate (EGCG), and quercetin. Many plants produce only small amounts of these secondary metabolites, but beneficial microbes associated with their host plant can increase their production.

The present invention contains bacteria that increase the production of such molecules. Indeed, the applicant noted that bacteria isolated from turmeric generate signal transduction networks in plants inducing a modulation of plant gene expression leading to the accumulation of certain bioactives in plant tissues.

The interaction of the microbiome with plants also makes it possible to increase the absorption of nutrients from the soil by plants, promoting their accumulation in plant tissues.

The present invention is therefore described as a plant treatment composition for use in increasing the beneficial properties on human health of cereals, fruits and vegetables.

Some molecules have low bioavailability and efficacy if ingested as it is and must undergo digestion by the intestinal microbiota to be activated. However, not all people have the bacteria necessary to activate polyphenols. For example, S-equol is formed by the digestion of daidzein by intestinal bacteria. S-equol has a higher anti-inflammatory efficacy than other isoflavones. Only 25 to 30% of the population have the bacteria necessary for this digestion. The bacteria making up the present invention have the genes and enzymes necessary for the transformation of daidzein into S-equol. By spraying the QKC composition on plants, the bacteria present in the composition digest daidzein into S-equol and therefore increase the anti-inflammatory efficacy of the plants on which the composition is used. The present invention is therefore described as a plant treatment composition for use in increasing the beneficial properties of cereals, fruits and vegetables on human health, characterized in that it increases the anti-inflammatory properties of cereals, fruits and vegetables.

Hyperglycemia (high blood sugar) means that there is too much sugar in the blood and is caused by the consumption of a diet high in sugar and low in fiber. Associated with diabetes, hyperglycemia can cause vomiting, excessive hunger and thirst, rapid heart rate, vision problems and other symptoms. Untreated hyperglycemia can lead to serious health problems as well as premature death.

Consumption of soluble dietary fiber reduces the rate of glucose absorption after the consumption of a high carbohydrate load due to its beneficial viscosity properties. By modulating the absorption of nutrients from the soil as well as modulating the gene expression of plants, the present invention modifies the nutritional qualities of the plants to which it is applied and in particular increases the proportion of fibers and resistant starch and decreases the quantity of sugars.

The present invention is therefore described as a plant treatment composition for its use in increasing the glycemia modulating properties by increasing the amount of dietary fiber and resistant starch in cereals, fruits and vegetables.

The present invention can be used in the form of liquids, foams, pastes, emulsions, oils, gel, jellies, syrups, solids, powders, sprays, aerosol.

The composition described in the present invention is sprayed or spread on the soil and/or the plant following the natural growth cycle of the plants. It can be used either in winter on the soil to improve the qualities of the soil and prepare for sowing, or during plant growth to stimulate plant growth.

### EXAMPLES

The present invention will be better understood on reading the following examples which illustrate the invention in a non-limiting manner.

### Example 1: List of QKC Metabolites

As an example, hereinafter is a non-exhaustive list of the metabolites present in a QKC type composition described by the present invention: jasmonic acid, coumarin, benzoazinoids, brassinosteroids, gibberellins, indole, pyrazine, catechin, octanoic acid, sitosterol, citric acid, phloretic acid, erucic acid, glucosamine, methyl jasmonate, lobeline, pyridoxine, trigonelline, syringic acid, procyanidins, geraniin, prodelphinidin, castalagin, punicalagin, tannic acid, resveratrol, apigenin-7-glucoside, luteorin, rutin, eriodictyol, kaempferol, phloretic acid, homovanillic acid, pelargonic acid, butyric acid, baicalin, saponarin, pyruvic acid, fumaric acid, hypoxanthine, spermine, ornithine, homovanillic acid.

### Example 2: Increased Productivity, Plant Growth and Nutritional Values

A study was conducted to evaluate the effect of QKC composition on the growth, productivity and nutritional values of rice plants. For this study, QKC and 3 other types of fertilizers were used on rice plants.

The fertilizers tested are:
GROUP 1: control—no fertilizer
GROUP 2: QKC
GROUP 3: Commercial composition of bacteria and fungi (yeasts)
GROUP 4: BIOCHAR (Rice bran charcoal obtained by pyrolysis of biomass)
GROUP 5: Biofertilizer composed of bacteria of the genus azobacter

In order to facilitate the understanding of the study, below are the definitions:
Grain yield: quantity by weight of grains produced in a given area
Biological yield: quantity by weight of biological matter produced on a given area
Spikelet: inflorescence containing the rice seed
Panicle: grouping of spikelets

The results on growth and productivity are presented in Table 1 below:

TABLE 1

| | Number of | | | Biological |
|---|---|---|---|---|
| | spikelets | Hollow | Grain yield | yield |
| TREATMENT | per panicle | spikelets | (kg/hectare) | (kg/hectare) |
| GROUP 1 | 119 | 6.37 | 3190.09 | 11975.34 |
| GROUP 2: | 170 | 5.17 | 4284.74 | 13931.29 |
| QKC | | | | |
| GROUP 3 | 135 | 5.96 | 3161.67 | 9568.33 |
| GROUP 4 | 128 | 5.83 | 3667.06 | 11690.39 |
| GROUP 5 | 136 | 6.62 | 3477.42 | 13204.09 |

It is observed that the number of spikelets per panicle, grain yield and biological yield are the highest with the treatment using the QKC composition. It is also observed that the number of hollow spikelets decreases with QKC. The QKC treatment increases the number of spikelets per panicle by 43%, grain yield by 34% and biological yield by 16% compared to the control.

In conclusion, the QKC composition is the most effective in increasing plant growth, fertility as well as increasing grain and biological yield.

The results of the effect of using QKC on rice composition are shown in Table 2 below:

TABLE 2

| | Nutritional values (per 100 g of rice grain) | | | | | |
|---|---|---|---|---|---|---|
| Treatment | Proteins | Carbohydrates | Fibres | Iron | Calcium | Magnesium |
| GROUP 1 | 5.7 g | 76.1 g | 3.4 g | 0.82 mg | 8.0 mg | 122 mg |
| GROUP 2: | 6.5 g | 74.5 g | 3.9 g | 0.92 mg | 8.5 mg | 127 mg |
| QKC | | | | | | |
| GROUP 3 | 5.5 g | 75.5 g | 3.3 g | 0.85 mg | 8.1 mg | 119 mg |
| GROUP 4 | 6.1 g | 76.1 g | 3.5 g | 0.84 mg | 8.0 mg | 118 mg |
| GROUP 5 | 5.3 g | 74.4 g | 3.2 g | 0.87 mg | 8.1 mg | 121 mg |

It is observed that with the QKC treatment, the protein, fiber, iron, calcium and magnesium values are higher compared to other treatments. Since magnesium has anti-inflammatory properties, it can be concluded that the anti-inflammatory properties of rice have been increased with the QKC treatment.

It is also observed that although the amount of fiber is higher, the amount of carbohydrates is lower, implying that the amount of sugars and starch has decreased.

In conclusion, the QKC treatment improved the nutritional values of rice and increased the amount of fiber in rice grains.

### Example 3: Increased Persistence of Bacteria in Soil

This study was conducted to evaluate the effect of the QKC composition on the persistence of bacteria in soil. For this study, lactic acid bacteria (LAB) either isolated (LAB group) or in a QKC composition (LAB in QKC) were inoculated into soil and their quantity was measured for 28 days.

Six seedling trays were each filled with one hundred grams of soil collected from a sugarcane field. Three trays were inoculated with the isolated lactic acid bacteria or in a QKC composition at a final soil concentration of 1010 CFU/g. The trays were placed in a plant growth chamber for 28 days. A 25 g sample was taken using a sterile spoon to determine the survivability of lactic acid bacteria at 0 days, 1 day, 3, 7, 14 and 28 days after inoculation.

The soil sample is serially diluted and then spread on an agar plate and incubated at 37° C. for 48 hours and the number of colonies counted. The quantity of bacteria is estimated by averaging the results found for the 3 seedling trays.

The results of the effect of QKC on the persistence of bacteria in the soil are presented in Table 3 below. The results are expressed in log CFU (colony forming unit) per gram of soil.

TABLE 3

| | 0 day | 1 day | 3 days | 7 days | 14 days | 28 days |
|---|---|---|---|---|---|---|
| BAL | 11.5 ± 0.5 | 10.2 ± 0.6 | 8.1 ± 0.7 | 7.7 ± 0.6 | 6.9 ± 0.5 | 6.2 ± 0.7 |
| BAL in | 11.4 ± 0.6 | 10.9 ± 0.4 | 10.5 ± 0.5 | 11.8 ± 0.7 | 12.1 ± 0.6 | 12.5 ± 0.6 |
| QKC | | | | | | |

The results show that without QKC, the number of bacteria decreases rapidly from the first day, probably due to competition with other bacteria present in the soil. On the contrary, with the QKC composition, the number of lactic bacteria increases by 1.1 log at the end of the study. This study clearly shows that the QKC composition allows the bacteria that compose it to have a long-term persistence in the soil.

### Example 4: Increase in the Anti-Inflammatory Properties of Plants

This study was carried out to evaluate the effect of the QKC composition on the anti-inflammatory properties of plants.

To do this, 2 orchards of the same variety of apple trees 1 km apart were sectioned. The QKC composition was sprayed on the apple trees of one of the orchards during flowering, while the other orchard received no treatment.

Apples were selected during the harvest and their anti-inflammatory properties were analyzed. For this, an extract of apples from the two orchards was made and tested on 40 overweight but healthy volunteers without distinction of sex, aged between 35 and 55 years, without any known disease. The 30 patients were randomly divided into 2 groups of 15 people. The first group ingested daily for 30 days 500 mg of the apple extract from the field vaporized with QKC and the second group ingested the apple extract from the placebo field. The participants underwent a blood test for inflammation markers (IL-6 and TNF-α) at the beginning and end of the study.

The blood level of IL-6 in the participants at the beginning and end of the study is shown in Table 4 below.

TABLE 4

| IL-6 (pg/ml) | Day 0 | Day 30 |
|---|---|---|
| QKC | 5.9 | 3.5 |
| Placebo | 6.0 | 4.8 |

It was observed that the blood level of IL-6 decreased significantly in both the QKC and placebo groups. However, the anti-inflammatory effect was more pronounced in the QKC group. The blood level of IL-6 decreased by 40.7% in the QKC group compared to a decrease of 20% in the placebo group.

The blood level of TNF-α in participants at the beginning and end of the study is shown in Table 5 below.

TABLE 5

| TNF-α (pg/ml) | Day 0 | Day 30 |
|---|---|---|
| QKC | 2.3 | 1.40 |
| Placebo | 2.1 | 1.6 |

It is observed that the blood level of TNF-α decreases significantly in both groups, however the decrease is higher in the QKC group. The blood level of TNF-α decreases by 39.1% in the Mito-boost group compared to a decrease of 23.8% in the placebo group.

This double-blind, placebo-controlled clinical study shows that QKC increases the anti-inflammatory properties of apples.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1: Plant treatment composition intended to increase the yield of agricultural production, preferably cereals, fruits and vegetables, and to increase their beneficial properties on human health, characterized in that it comprises a mixture of live bacteria originating from the microbiota of the rhizomes of turmeric (Curcuma longa), preferably turmeric originating from Bhutan, a lysate of said bacteria and metabolites originating from the fermentation of these same bacteria.

2: Plant treatment composition according to claim 1, characterized in that the composition comprises one or more strains of bacteria taken from the following list: Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, Bradyrhizobium elkanii, Bradyrhizobium japonicum, Gluconacetobacter diazotrophicus, Azospirillum brasilense.

3: Plant treatment composition according to claim 1 or claim 2, said composition being in the form of liquids, foams, pastes, emulsions, oils, gel, jellies, syrups, solids, powders, sprays, or aerosols.

4: A method of manufacturing the plant treatment composition according to claim 1 or claim 2, comprising the steps of:
isolating bacteria from rhizomes of Curcuma longa, preferably from a plantation located in Bhutan and not using chemical fertilizers or herbicides; and
inoculating at least one substrate with said bacteria and fermenting the mixture of bacteria with said at least one substrate.

5: Use of the plant treatment composition according to any one of claims 1 to 3, for increasing soil fertility by increasing the concentration of nutrients and decreasing the amount of toxic substances.

6: Use of the plant treatment composition according to any one of claims 1 to 3, for increasing the yield of cereals, fruits and vegetables, by increasing the growth of plants, their fertility and by increasing the number and weight of seeds, fruits and vegetables.

7: Use of the plant treatment composition according to any one of claims 1 to 3, for increasing the beneficial properties on human health of cereals, fruits and vegetables.

8: Use of the plant treatment composition according to any one of claims 1 to 3, for increasing the anti-inflammatory properties and the blood sugar regulating properties of cereals, fruits and vegetables.

9: Use of the plant treatment composition according to any one of claims 1 to 3, for increasing the dietary fiber content of cereals, fruits and vegetables.

10: A method for increasing the yield of at least one plant, wherein the composition according to any one of claims 1 to 3 is sprayed or spread on the soil and/or the at least one plant.

11: A plant treatment composition comprising a mixture of bacteria originating from the microbiota of the rhizomes of Curcuma longa, a lysate of said mixture of bacteria, and metabolites originating from the fermentation of said mixture of bacteria.

12: The plant treatment composition of claim 11, wherein said mixture of bacteria is selected from one or more of the group consisting of Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, Bradyrhizobium elkanii, Bradyrhizobium japonicum, Gluconacetobacter diazotrophicus, and Azospirillum brasilense.

13: A method of preparing a plant treatment composition, which comprises:
isolating a mixture of bacteria from rhizomes of Curcuma longa; and
inoculating at least one substrate with said mixture of bacteria and fermenting said mixture of bacteria with said at least one substrate.

14: A method of increasing the yield of agriculture products, which comprises:
isolating a mixture of bacteria selected from one or more of the group consisting of Lactobacillus fermentum, Lactobacillus casei, Lactobacillus plantarum, Lactobacillus acidophilus, Bifidobacterium longum, Bifidobacterium bifidum, Bifidobacterium adolescentis, Bifidobacterium infantis, Lactobacillus brevis, Lactobacillus Jensenii, Lactobacillus paracasei, Christensenella minuta, Lactiplantibacillus plantarum, Lactiplantibacillus paraplantarum, Mycobacterium vaccae, Pseudomonas japonica, Pseudomonas fluorescens, Archaeospora trappei, Methylobacterium crusticola, Bradyrhizobium elkanii, Bradyrhizobium japonicum, Gluconacetobacter diazotrophicus, and Azospirillum brasilense;
inoculating at least one substrate with said mixture of bacteria and fermenting said mixture of bacteria with said at least one substrate to produce a fermented mixture; and
applying said fermented mixture to soil of said agriculture products.
